(19) United States
(12) Patent Application Publication
(10) Pub. No.: US 2010/0143890 A1
(43) Pub. Date: Jun. 10, 2010

(54) PEPTIDE COMPOUNDS FOR CAPTURING OR INHIBITING AVIAN INFLUENZA VIRUS AND APPLICATION THEREOF

(75) Inventors: Hyobong HONG, Daejeon-city (KR); Soohyung Lee, Daejeon-city (KR); Taewan Kim, Daejeon-city (KR); Myungae Chung, Daejeon-city (KR); Sungwon Shon, Daejeon-city (KR)

Correspondence Address: STAAS & HALSEY LLP, SUITE 700, 1201 NEW YORK AVENUE, N.W., WASHINGTON, DC, 20005, US

(73) Assignee: ELECTRONICS AND TELECOMMUNICATIONS RESEARCH INSTITUTE, Daejeon-city (KR)

(21) Appl. No.: 12/633,131

(22) Filed: Dec. 8, 2009

(30) Foreign Application Priority Data
Dec. 8, 2008 (KR) 10-2008-0124130
Oct. 8, 2009 (KR) 10-2009-0095680

(51) Int. Cl. C12Q 1/70; C12M 1/34; C07K 14/00; C07D 235/02

(52) U.S. Cl. 435/5; 435/287.1; 530/350; 548/304.1

(57) ABSTRACT

Disclosed herein are peptide, particularly dipeptide compounds and the application thereof to the detection or inhibition of AI virus. The peptide compounds are more stable and easier to synthesize and store than are antibodies. In addition, having strong binding forces for the H5 protein of AI virus, the peptide compounds are useful as capturers or inhibitors of AI virus.

### RELATED APPLICATIONS

The present application claims priority to Korean Patent Application Serial Number 10-2008-0124130, filed on Dec. 8, 2008 and Korean Patent Application Serial Number 10-2009-095680, filed on Oct. 8, 2009, the entirety of which are hereby incorporated by reference.

### BACKGROUND OF THE INVENTION

1. Field of the Invention

The present invention relates to a peptide compound for the capture or inhibition of avian influenza virus and the application thereof.

2. Description of the Related Art

Avian influenza (hereinafter referred to as “AI”) virus has recently done great damage to the domestic poultry industry as a result of the extent of damage having expanded and the number of outbreaks having increased. Although no cases of human AI infection have been reported in Korea thus far, it may result in fatal damage and in fact has led to 93 infection cases and 50-60% of the mortality rate of highly pathogenic H5N1 in Vietnam. For this reason, international organizations, such as the WHO, are paying great attention to AI.

Conventional techniques related to the AI virus include a recombinant adenovirus vector carrying an AI antigen, an immune composition comprising the recombinant vector and adenovirus, and immunization against AI or an epidemic influenza by inoculation with the immune composition, etc. Additionally, there are methods of preparing a peptide-based anti-influenza vaccine against influenza A and B, and PDMS-based, anti-virus early-warning systems for detecting the highly pathogenic AI. These techniques are related to just a pharmaceutical uses such as a vaccine, without instructions being provided about the uses thereof in developing AI virus-capturing materials or biosensors.

Although several therapeutics (e.g., Tamiflu) or vaccines against AI have already been developed, there are no particular prevention measures upon AI infection in poultry, except for the closing of a region where there was an outbreak or the slaughter of infected poultry. Hence, currently the best solution to AI virus problems is to detect AI virus infection as early as possible so as to take rapid countermeasures.

Examples of fundamental techniques associated with AI detection or measurement include 1) a simple diagnostic kit, 2) egg inoculation, and 3) RT-PCR (Real Time Polymerase Chain Reaction). As concerns the simple diagnostic kit, the production of a specific antibody is a core technique. In the case of the egg inoculation or RT-PCR, the proliferation of specific genes of AI virus accounts for the core technique therefor. So far, the development of ligands specific for AI virus has been focused on virus inhibitors (therapeutics) while most biosensors are directed to antibodies. Usually based on ELISA (Enzyme-Linked ImmunoSorbent Assay), simple diagnostic kits are too expensive for ordinary livestock farmers to utilize. In addition, antibodies, which commonly play pivotal roles in simple diagnostic kits, require that very difficult storage conditions or usage conditions be followed. Also, antibodies themselves are apt to denature, imparting limitations to the use of simple diagnostic kits in the field, these usually being conditions which are more severe than those of laboratories.

### SUMMARY OF THE INVENTION

Accordingly, the present invention has been made keeping in mind the above problems occurring in the prior art, and an object of the present invention is to provide a capturing compound which can be used in lieu of antibodies to detect an AI virus, which is economically profitable compared with antibodies and can be stored for a long period of time.

It is another object of the present invention to provide an inhibiting compound which can strongly bind to proteins of the AI virus to suppress the activity of the AI virus.

It is a further object of the present invention to provide an agent for capturing or inhibiting AI virus, comprising the compound as an active ingredient.

It is still a further object of the present invention to provide a method for detecting the AI virus, comprising the use of the capturing compound.

It is still another object of the present invention to provide a biosensor for diagnosing AI infection, which comprises the capturing compound as an active ingredient.

In accordance with an aspect thereof, the present invention provides a compound for capturing or inhibiting AI virus, represented by the following Chemical Formula 1:

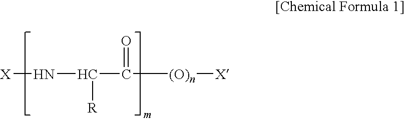

wherein

X and X′ are independently H; a functional group selected from the group consisting of biotin, streptavidin and avidin; or a functional moiety composed of a functional group selected from the group consisting of biotin, streptavidin and avidin, and a linker which connects the functional group to the backbone of the compound of Chemical Formula 1 therethrough;

m is an integer of 2˜10;

n is 0 or 1; and

R is selected from the group consisting of —H, —CH₃, —CH(CH₃)₂, —CH₂CH(CH₃)₂, —CHCH₃CH₂CH₃, —CH₂OH, —CHOHCH₃, —CH₂SH, —(CH₂)₂SCH₃, —CH₂COOH, —CH₂CONH₂, —(CH₂)₂COOH, —(CH₂)₂CONH₂, —(CH₂)₃CH₂NH₂, —(CH₂)₃NHCNHNH₂,

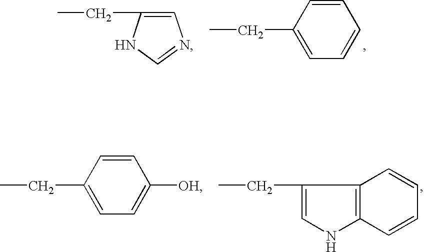

—CH₂CH₂CH₂— (proline) and —CH₂SSCH₂— (cystine).

In accordance with another aspect thereof, the present invention provides an agent for capturing and inhibiting AI virus, comprising the compound of Chemical Formula 1.

In accordance with a further aspect thereof, the present invention provides a method for detecting AI virus, comprising bringing the compound of Chemical Formula 1 into contact with a sample to be tested.

In accordance with still a further aspect thereof, the present invention provides a biosensor comprising the compound of Chemical Formula 1 as a capturer of the AI virus.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view showing a molecular structure of a compound for capturing or inhibiting AI virus in accordance with the present invention;

FIG. 2 is a schematic diagram showing a streptavidin-coated bead the surface of which is modified by conjugation with a compound for capturing or inhibiting AI virus in accordance with the present invention;

FIG. 3 is a schematic diagram showing in a stepwise manner the preparation of a biosensor using a compound for capturing and inhibiting AI virus in accordance with the present invention;

FIG. 4 shows affinities of 29 amino acid dimers, selected in Example 1, for the H5 protein of AI virus in terms of free energy measured by use of Flexx-p (BiosolveIT, Germany);

FIG. 5 is a photograph showing the results of FIG. 4

FIGS. 6A and 6B show the results of the comparison experiment of binding properties with antibody which is commercially available [Ab: antibody, NR1: sialic acid, NR2: 3-silaiic-lactose, NR3: 6′-sialic lactose, SM1: YA (single word of the amino acid dimer), SM2: QH, SM3: IL]

FIGS. 7A and 7B show the results of experiment using real chicken blood sample of chicken red serum with antigen, concentration: 6.7 picoM, SE: sample of chicken red serum).

FIG. 8 is a graph showing analysis results obtained in Example 2.

### DESCRIPTION OF THE PREFERRED EMBODIMENTS

In accordance with an aspect thereof, the present invention pertains to a compound for capturing or inhibiting AI virus, represented by the following Chemical Formula 1:

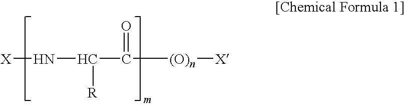

wherein

X and X′ are independently H; a functional group selected from the group consisting of biotin, streptavidin and avidin; or a functional moiety composed of a functional group selected from the group consisting of biotin, streptavidin and avidin, and a linker which connects the functional group to the backbone of the compound of Chemical Formula 1 therethrough;

m is an integer of 2˜10;

n is 0 or 1; and

R is selected from the group consisting of —H, —CH₃, —CH(CH₃)₂, —CH₂CH(CH₃)₂, —CHCH₃CH₂CH₃, —CH₂OH, —CHOHCH₃, —CH₂SH, —(CH₂)₂SCH₃, —CH₂COOH, —CH₂CONH₂, —(CH₂)₂COOH, —(CH₂)₂CONH₂, —(CH₂)₃, —(CH₂)₃NHCNHNH₂,

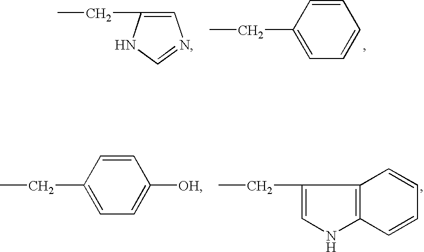

—CH₂CH₂CH₂— (proline) and —CH₂SSCH₂— (cystine).

Examples of the linkers useful in the present invention include polyethylene glycols (PEGs), DNA, alkylene of C₁˜C₂₀, 1-ethyl-3-(3-dimethylaminopropyl)-carbodiimide (EDC), carbonyldiimidazole (CDI), Sulfo-NHS (Sulfosuccinimidyl), isocyanate derivatives, acylazide derivatives, N-hydroxysuccinimide (NHS), sulfonyl chloride derivatives, aldehyde derivatives, epoxy derivatives, etc.

In greater detail, the linker suitable for X′ may include 1-ethyl-3-(3-dimethylaminopropyl)-carbodiimide (EDC), carbonyldiimidazole (CDI), and Sulfo-NHS (Sulfosuccinimidyl), while isocyanate derivatives capable of forming isothiourea bonds, acylazide derivatives capable of amide bonds, N-hydroxysuccinimide (NHS), sulfonyl chloride derivatives capable of forming sulfonamide bonds, or aldehyde derivatives or epoxy derivatives capable of forming secondary amide bonds may be used as a linker for X.

The compound of Chemical Formula 1 may be structurally changed by modifying the amine group of the liker or the peptide with an arylating agent, an imidoester, EDC, an anhydride, a fluorophenyl ester, etc.

The compound represented by Chemical Formula 1 binds to the H5 protein of AI virus so as to capture or inhibit AI virus.

Preferably, the compound for capturing the epitopes of H5N1 HA or inhibiting AI virus, represented by Chemical Formula 1, may be based on a dipeptide compound (m=2) selected from the group consisting of ALA-ALA, ARG-ARG, ARG-ASN, ASN-TYR, CYS-VAL, GW-HIS, GLN-ILE, GLU-HIS, GLU-LYS, GLY-GLU, ILE-THR, LEU-ALA, LYS-LYS, MET-ALA, MET-APS, PHE-ASN, PRO-ALA, SER-ARG, SER-CYS, SER-LYS, SER-VAL, THR-GLN, THR-GLU, THR-LYS, TRP-ARG, TYR-ALA, VAL-ALA, VAL-HIS and GLU-HIS.

In particular, the compound for capturing the epitopes of H5N1 HA or inhibiting AI virus, represented by Chemical Formula 1, may be based on a dipeptide compound selected from the group consisting of GL-HIS, TYR-ALA, ALA-ALA, CYS-VAL, SER-LYS, VAL-ALA, GLU-HIS, LEU-ALA, ARG-ARG, SER-VAL, TRP-ARG, ASN-TYR, SER-CYS; preferably GLN-HIS or TYR-ALA, when the epitope has the sequence of RNSPQRERRRKKRG.

In particular, the compound for capturing the epitopes of H5N1 HA or inhibiting AI virus, represented by Chemical Formula 1, may be based on a dipeptide compound selected from the group consisting of Ile-THR, ASN-TYR, TYR-ALA, THR-LYS, TRP-ARG, GLU-LYS, ALA-ALA, PHE-ASN, GLN-ILE, LYS-LYS, SER-CYS, SER-LYS, LEU-ALA; preferably ILE-THR or ASN-TYR when the epitope has the sequence of CYPGDFNDYEELKHL.

The compound represented by Chemical Formula 1 binds to the H5 protein of AI virus so as to capture or inhibit AI virus.

In the compound of Chemical Formula 1, m is preferably an integer of 2˜5, more preferably an integer of 2 or 3 and most preferably an integer of 2.

Also, the present invention pertains to an AI virus-capturing or -inhibiting agent, comprising the compound of Chemical Formula 1 as an active ingredient.

The AI virus-capturing or -inhibiting agent may further comprise a pharmaceutically acceptable carrier. As used herein, the term “pharmaceutically acceptable carrier” is intended to refer to a medium applicable to administration as exemplified by solvents, dispersion media, isotonic solutions, absorption delaying agents, etc. The media suitable for use in the administration of active medicinal ingredients are well known in the art. Auxiliary active compounds may be included in the inhibitor.

Also, the present invention pertains to a complex for capturing AI virus, comprising an AI virus-capturing or -inhibiting agent comprising the compound of Chemical Formula 1; a support; and an immobilizer for fixing the AI virus-capturing or -inhibiting agent on the support.

As the support, a substrate such as silicon wafer, metal, glass, quartz, etc. or magnetic beads in nano- or micro size may be used. Additionally, a carbon nanotube may be available as the support.

Also, the present invention pertains to a method for detecting AI virus, comprising contacting the compound of Chemical Formula 1 with a sample from a subject.

In the method, the contacting step comprises fixing either the compound of Chemical Formula 1 or the sample on a substrate and bringing the compound of Chemical Formula 1 into contact with the sample when it is fixed on the substrate and vice versa.

In this case, the contacting step may further comprise adding a label-conjugated secondary capturing material to the substrate when the sample is brought into contact with the compound of Chemical Formula 1 after the compound of Chemical Formula 1 is fixed on the substrate.

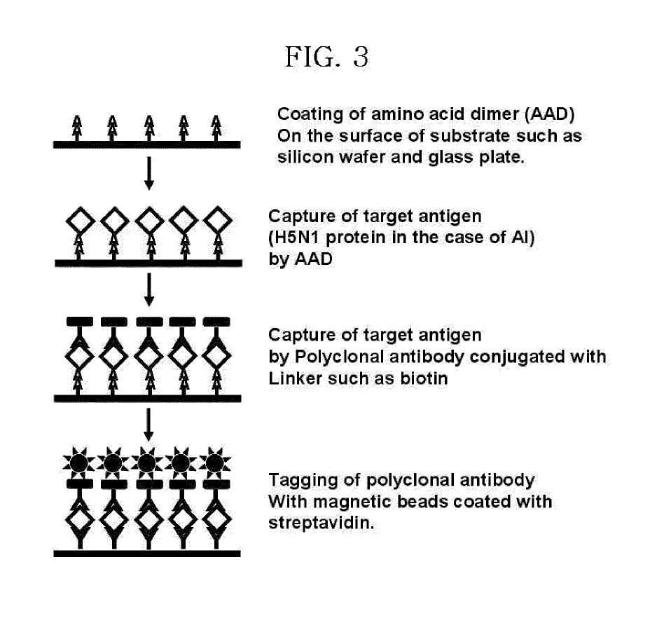

This method is a modification of a sandwich immunoassay as schematically illustrated in FIG. 3.

So long as it is typically accepted in the art, any substrate may be used without any limitation. Examples include a silicon wafer, metal, glass, quartz, etc., but are not limited thereto. In the present invention, the secondary capturing material is intended to mean a material such as a polyclonal antibody which can bind to AI virus captured by the compound of Chemical Formula 1.

In the method, the contacting step may comprise fixing the compound of Chemical Formula 1on nano- or microbeads; and bringing the sample into contact with the fixed compound of Chemical Formula 1.

In the method, the contacting step may further comprise adding a label-conjugated secondary capturing material to the nano- or microbeads after the sample is brought into contact with the fixed compound of Chemical Formula 1.

No particular limitations are imparted to the nano- or microbeads so long as they are typically used in the art. Preferable are magnetic particles or carbon nanotubes.

Reference now should be made to the drawings to describe the present invention in more detail.

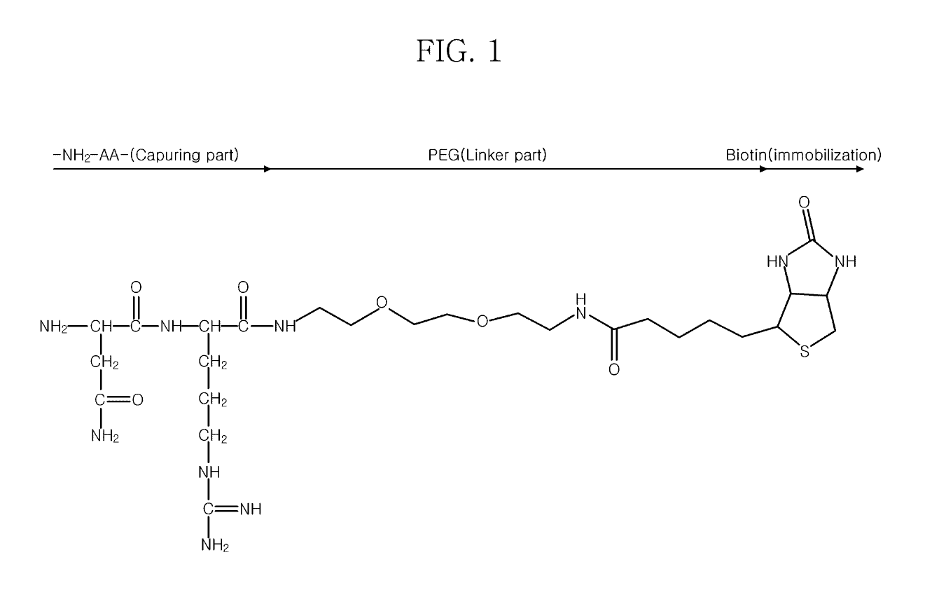

With reference to FIG. 1, a compound, which is an example of Chemical Formula 1 and is used for capturing or inhibiting AI virus, is shown along with the analyzed molecular structure thereof. As can be seen from the molecular structure, the compound consists of AsnArg as a capturing part (—NH₂-AA), PEG as a linker part and biotin as an immobilization part. The amino acid dimer AsnArg was found to have the highest affinity for AI virus as measured theoretically and by an affinity assay. Instead of PEG and biotin, other structures may be used for the linker part and the immobilization part, respectively. For instance, alkyl structures with different lengths, or DNA may occupy the linker part while a certain functional group such as —NH₂, —COOH— or —OH may be responsible for the immobilization part.

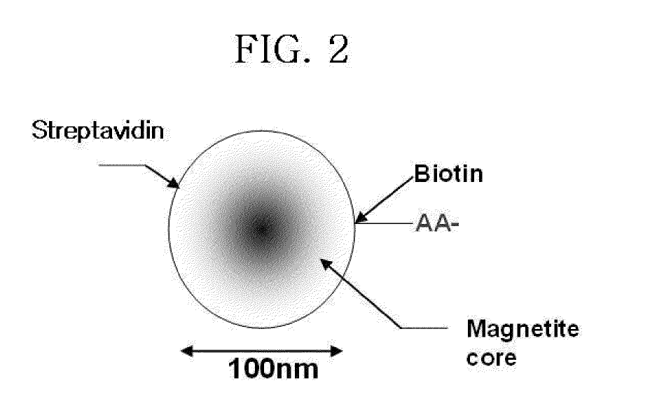

With reference to FIG. 2, a streptavidin-coated bead, modified with a biotin-conjugated amino acid dimer, used to separate the H5 antigen of AI virus from a sample, is shown.

Turning to FIG. 3, the preparation of a biosensor with a compound for capturing or inhibiting AI virus in accordance with the present invention is illustrated in a stepwise manner.

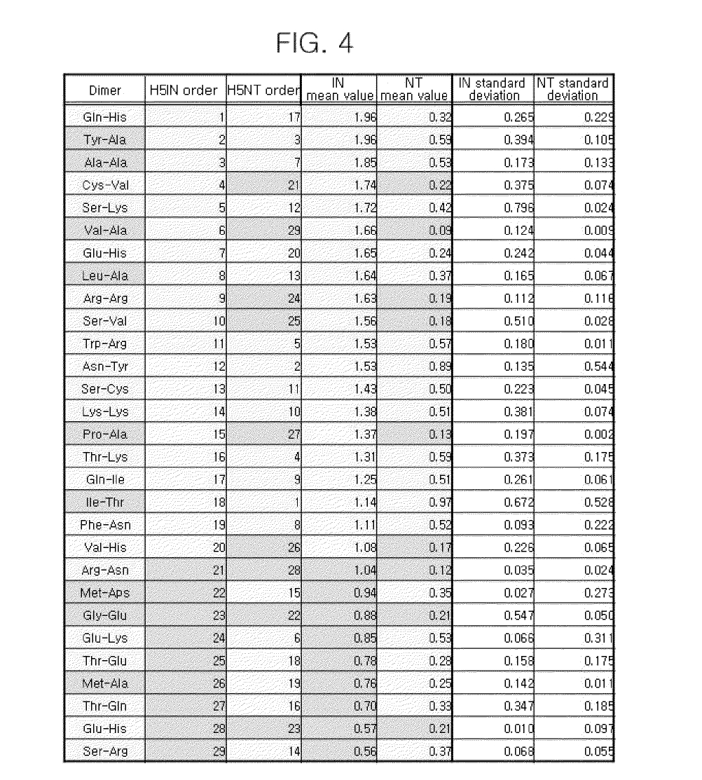

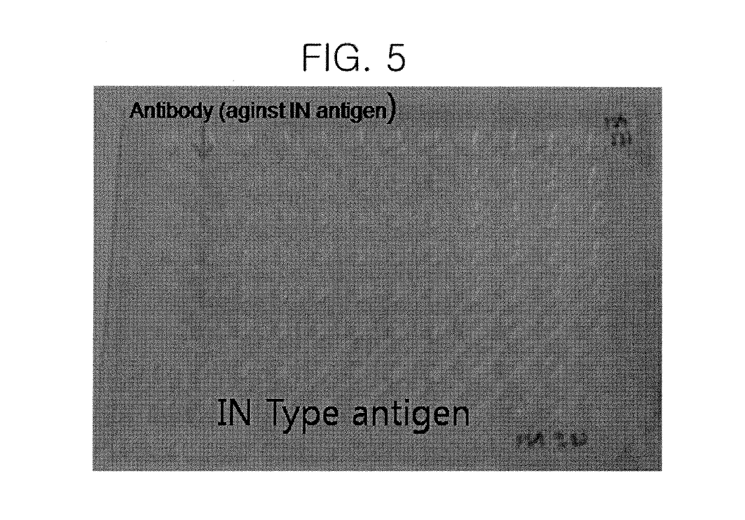

With reference to FIGS. 4 and 5, FIG. 4 shows affinities of 29 amino acid dimers, selected in Example 1, for the H5 protein of AI virus in terms of free energy measured by use of Flexx-p (BiosolvelT, Germany). FIG. 5 is a photograph showing the results of FIG. 4. As is apparent from the data of the Table of FIG. 4, the capturing compounds prepared from the 29 selected amino acid dimers are different in binding properties from each other, indicating that the peptides, although very short in length, show different binding properties with regard to antigens.

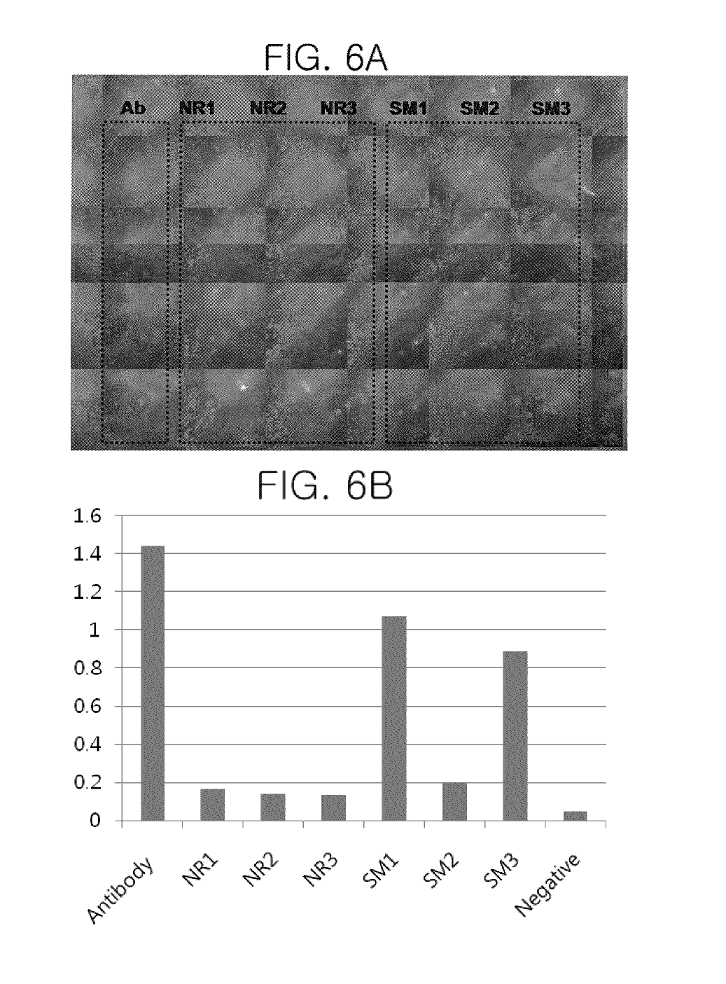

With reference to FIGS. 6A and 6B, the FIGS. 6A and 6B show the comparison experiment results of binding properties with antibody which is commercially available. In this experiment, 2 types of NT and IT as an antibody were used and 3 lines in the middle of them (which are not active) are sialic acid (NR1), 3-silaiic-lactose (NR2) and 6′-sialic lactose (NR3) known as receptor of H5 protein exist in nature. This experiment results shows that newly developed amino acid dimmers have strong binding properties rather than the receptor existing in nature (generally known to participate in infiltration on surface of the cell of AI virus; J. Gen. Viol 2004.85:100 1-5).

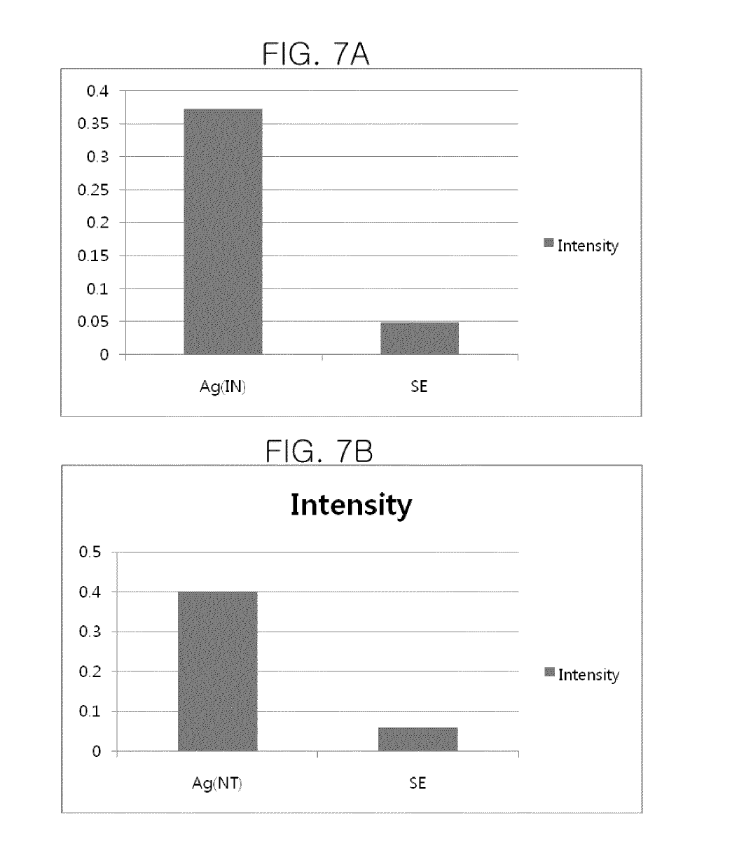

With reference to FIGS. 7A and 7B, the FIGS. 7A and 7B show the experiment results using real chicken blood. In the experiment using QH (Single word) of an amino acid dimmer as a capturing material, the blood of health chicken (SE: sample of chicken red serum) did not reaction with QH, but, the sample containing AI virus (Ag: sample of chicken red serum with antigen, concentration: 6.7 picoM) did reaction with QH. With this result, we found that the amino acid dimmers of the present invention selectively react with the antigen of AI virus.

A better understanding of the present invention may be obtained through the following examples which are set forth to illustrate, but are not to be construed as limiting the present invention.

### Example 1

### Preparation of Dipeptides

For use in capturing AI virus proteins or peptides, the compound of the present invention was prepared through the following steps:

Step 1: Structural analysis of AI virus proteins or peptides to be targeted;

Step 2: Based on the analyzed structure, computational chemistry is used to model a variety of ligands which are expected to bind to the target proteins or peptides and suggest thermodynamically stable compounds upon virtually binding them to the target proteins or peptides;

Step 3: From the suggestions of Step 2, appropriate compounds are selected and synthesized, with the exclusion of compounds which are impossible to practically synthesize or which are already known about;

Step 4: The binding of the synthetic compounds of Step 3 to AI virus proteins is tested and they are remodeled into forms which are applicable to sensors; and

Step 5: Binding of the remodeled compounds to the AI virus proteins (3425P and 3427P, commercially available from ProSci Inc., U.S.A.) is tested to determine whether they can be used as detectors of AI virus.

In Step 1, a part of peptides rather than whole protein in the commercially available peptides are used for the antigen. Also, the cases in which a part of peptides are used for the antibody are increasing.

In Step 2, binding properties of various compounds virtually synthesized on a computer was confirmed and In Step 3, dipeptides which were practically synthesizable and obtainable at low expense were selected and synthesized.

In the remodeling of Step 4, the linker and the anchor, both used to apply the designed dipeptides to a sensor, may change the binding properties of the designed peptides. The binding properties of the remodeled compounds were therefore analyzed using practical AI virus antigens in Step 5.

However, because AI virus protein cannot be used for it's stability and legal issues, antigenic materials available from a world-wide authorized company, such as those identified as ProSci 3425P and 3427P provided by ProSci Inc. U.S.A., were used to test the binding properties of the capturing compounds.

In the binding test of Step 5, it is very important to find the remodeled compounds which show such selectivity that they react with target antigens, but do not bind to the other proteins present in the body. In this example, hence, binding properties of the remolded compounds were tested not only to target antigens but also to the healthy chicken red sera.

The new compounds for capturing or inhibiting AI virus, provided through the procedure in accordance with the present invention, are peptidyl materials with low-molecular weights, which are based on two to ten amino acids, preferably two to five, more preferably two or three and most preferably two amino acids.

1. Selection of Amino Acid dimers

1) Reference was made to the RCSB protein data bank data (PDB ID NO 1JSN, 1JSM, 3 GBM, 3FKU, 2IBX, 2FKO and etc) concerning the structures of target proteins (antigens), and the sequences of practically used antigens were ordered from ProSci, U.S.A.

2) Water molecules were removed from the obtained proteins using a freeware program called Vega ZZ (http://nova.colombo58.unimi.it) and the water-free target proteins were subjected to virtual association with hydrogen molecules. However, some water molecules were not removed from the models because of their important role such as stabilization of the ligand-receptor interaction.

3) Affinity between the known 400 amino acid dimers and H5 protein was calculated (the lower the calculated value is, the more stable the bonding between the amino acid dimer and, H5 protein is, because it is free energy).

4) Excellent virtual binding properties made selection of 36 among the 400 amino acid dimmers and they were ordered with 95% or higher purity from SeouLin Bioscience Inc., Korea (synthesized in Thermo Science) and/or Peptorn Inc, Korea.

2. Preparation of Capturing Compounds

1) Selected amino acid dimers were diluted to 1.0 mM in PBS.

2) Each of the amino acid dimer solutions (1.0 mM) were mixed with one volume of a 1.0 mM NHS-sulfo-biotin solution (PBS) to prepare a 0.5 mM amino acid dimer solutions.

3) The 0.5 mM amino acid dimer solutions were incubated at 30° C. for 30 min in an Eppendorf shaking reactor.

4) Again, the 0.5 mM solutions were 50-fold diluted, that is, to 10 μM, which was determined in consideration of the fact that the antibodies provided from ProSCI were about 6.7 μM.

5) The solutions thus prepared were stored in a refrigerator until use without ion removal.

Hereinafter, “capturing compounds” is a term used to refer to amino acid dimers conjugated with a linker+anchor complex (the linker+anchor complex means Sulfo-NHS-Biotin provided from Pierce U.S.A.).

3. Coating of Antigen

1) The microplates used in this experiment were NUNC Maxisorp® microplates. Microplates were used in their original, new state without being washed.

2) For use, the antigens were 1/1000 diluted in a coating buffer (e.g., 10 μL of the antigen solution was mixed with 10 mL of coating buffer).

3) Chicken red serum (CRS) was 1/10 diluted in a coating buffer. After 3 hours, the gore of the extracted chicken red serum is removed using a centrifugal separator. And only plasma ingredients were used. And, 0.02% of thimersol is added in order to ensure long preservation and kept in frozen.

4) The chicken red serum (CRS) was 1/10 diluted in a coating buffer, because, when the CRS are used as a negative reference, although concentration of the total protein may be higher than that of the antigen, contents of individual protein are small.

5) As for the spiked solution (expressed usually as SP on microplates), it was prepared by mixing 50 μL of an antigen stock with 950 μL of CRS in the first experiment. For practical use, it was 1/10 diluted in coating buffer. However, in the experiment dated January 14^{th}, coating buffer 1.8 ml, CRS 180 ul, antigen solution 20 ul were added just before the experiment. Therefore, ratio is set to same.

4. Binding Test with Antigen

1) The prepared antigen was seeded in an amount of 100 μL per well on microplates and incubated overnight at 20˜23° C. (room temperature) in the humidity chamber.

2) Typically, each well of antigen-coated plates was washed twice or three times with autoclaved, deionized water and incubated for 2 hrs at 20° C. with 200 μL of blocking buffer. However, it is skipped in the CRS case. 3) The blocking buffer used in this binding test comprised 0.1% BSA and 0.02% thimersol in PBS, pH 7.2 and was not autoclaved. A blocking step, if conducted, was followed by washing three or more times with washing buffer and then twice or more times with PBS.

4) After completion of the washing, the plates were turned upside down and tapped three times against blotting paper so that no residue was left on the plates.

5) Thereafter, the solutions prepared in section 1 (Preparation of biotinlated dimer) were allocated in an amount of 100 μL. At this time, as a positive reference, commercially available antibody (avian influenza A hemagglutinin antibody CATALOGU No 3425 & 3427 commercially available from ProSci Inc.) is allocated in an amount of 100 μL. And, the sample to which an antibody or the newly developed amino acid dimmer has not been added was used as a negative reference (other conditions are the same). 6) They were incubated at room temperature for 1 hr with shaking at 100 rpm.

7) Washing was conducted three to five times with a washing buffer and then three to four times with PBS.

8) Step 5) was repeated.

9) Streptavidin-HRP (Horse radish peroxides) was 1/10,000 diluted in PBS and allocated in an amount of 100 μL in each well.

10) The washing of Step 2) was repeated.

11) The procedure of Step 5) was repeated.

12) 50 μL of a mixture of 1:1 of TMB:substrate solution was allocated to the prepared solution.

13) Incubation was conducted at 37° C. for 15 min. At this time, it is most important that the reaction time is accurately kept.

14) After 15 min, a stop solution (2N HCl) was allocated, and then within 10 min, absorbance was measured at 450 nm using a microplate reader.

### Example 2

### Binding of a Selected Amino Acid Dimer (AA) to H5 Protein

2-1. Experiment with Magnetic Beads

In this experiment, magnetic beads conjugated with amino acid dimers functioned to separate the H5 protein from samples.

1) The H5 protein of AI virus was purchased from Bioassay Systems (USA, CA) (Catalog: Birdflu (H5HA-EAB)).

2) First, using NHS-Sulfo-biotin, biotin was conjugated to the NH₂ of each of the 36 amino acid dimers selected in Example 1.

3) The NHS-Sulfo-biotin was used in the same molar amount as the amino acid dimer. For example, when an amino acid dimmer was used in an amount of 0.1 micro mole, one micro mole of NHS-sulfo-biotin was employed.

4) After the solution of the streptavidin-coated bead of which size is 100 nm was washed three time with PBS, to the solution was added the synthesized amino acid dimer-PEG-biotin (36 samples) so that the surface of the streptavidin-coated beads was coated with the amino acid dimers (FIG. 2).

5) The 100 nm beads were purchased from Chemicell, Germany. They were washed three times with PBS before being mixed with the same volume of the biotin-conjugated amino acid dimer solution. In this example, the solutions were used in an amount of 50 microliters respectively.

6) The 36 bead solutions thus obtained (different from each other in the amino acid dimer conjugated to the bead surface) were washed three times with PBS to remove excess reagents.

7) The H5 protein was diluted 1/100 in PBS and 100 μL of the dilution was added to the 36 bead solutions.

8) The samples thus prepared were incubated for 2 hours at room temperature.

9) After the incubation, beads were separated using a magnet, and the supernatant was analyzed for protein quantity.

2-2 Experiment with Substrate (Silicone Oxide Film Such as Silicon Wafer, Glass and Quartz) on which the AI Virus-Capturing Amino Acid Dimer was Immobilized

1) A silicon wafer (Nano Fab Center, Daejeon, Korea) on which silicone oxide was grown to a length of 10 nm was cut into dimensions 6 mm wide and 8 cm long.

2) The silicon wafer pieces were cleaned by an RCA or a plasma procedure to remove organic matter therefrom.

3) A solution of MeOH:HCl (1:1) was used to impart silanol groups to the wafer surface.

4) The wafer was further treated with 10% PEI solution (in 50 mM, calcium carbonate, pH 8.0) to form free amines on the surface.

5) NHS-sulfo-biotin, commercially available from Pierce, or a corresponding bi-functional compound was selectively immobilized on the substrate in such a manner that N-hydroxysuccimide was bonded to the free amine on the substrate while the biotin moiety was free.

6) The coated substrate was treated with a solution of streptavidin in PBS (pH 7.4), followed by incubation at room temperature for 0.5˜1 hour to fix the streptavidin.

7) A solution of the amino acid-polyethylene glycol-biotin (AA-PEG-Biotin), prepared in Example 2-1, 1)-4), in PBS was applied to the silicon wafer, followed by incubation at room temperature for 30 min. Afterwards, the compounds remaining unreacted were removed using PBS (containing 1% Tween 20).

8) Subsequently, a solution of H5 antigen was applied to the silicon wafer prepared in 7) and incubated at room temperature for 2 hours.

9) Then, the substrate was treated with a biotin-conjugated secondary antibody (in PBS) to form a sandwich structure for ELISA.

10) Fluidmag-BC-biotin (Size: 100 nm, concentration: 10.0 mg/ml), commercially available from Chemicell, Germany, was diluted 1/100 in PBS before being used as magnetic nano-beads in this experiment.

11) An examination was made of whether the streptavidin-coated beads (magnetic nano-beads) selectively bonded to the substrate prepared in 9) (see FIG. 3). At this time, the beads accounted for the role of HRP in ELISA.

12) Compounds remaining unreacted were removed using PBS after which signals for labeled beads were measured using a magnetic reader.

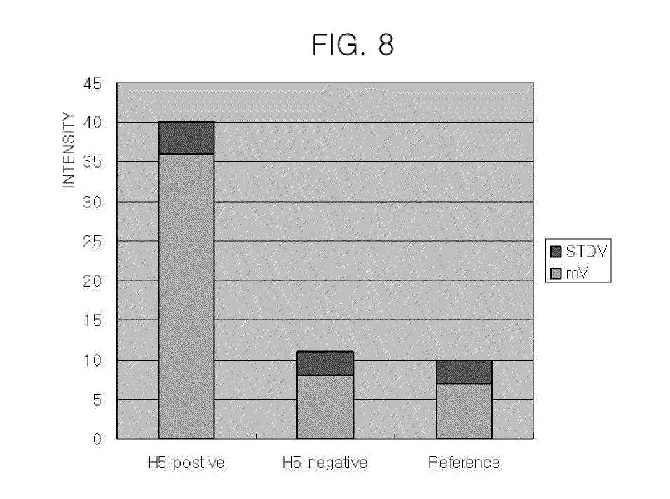

The results from the use of NH₂-AsnArg-COOH are depicted in FIG. 8.

As described hitherto, having an ability to strongly bind to AI virus, as described hitherto, the peptide compounds in accordance with the present invention show excellent activity of capturing or inhibiting AI coronavirus in addition to being provided at low cost thanks to the ease of their synthesis. Also, the peptide compounds are so stable that they are not destroyed even after one year of storage. The compounds of the present invention thus overcome the problems generated by the immune analysis using an antibody, such as low activity or degradation of antibodies, difficulty in analysis at poorly equipped labs, the sacrifice of a variety or numerous animals for producing antibodies, etc. In contrast to antibodies, further, the peptide compounds for capturing or inhibiting AI virus in accordance with the present invention can be readily applied to biosensors.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An agent for capturing or inhibiting avian influenza virus, comprising as an active ingredient a compound represented by the following Chemical Formula 1: 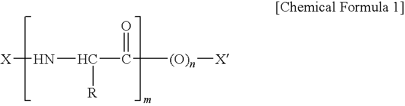
wherein
X and X′ are independently: H; a functional group selected from the group consisting of biotin, streptavidin and avidin; or a functional moiety composed of a functional group selected from the group consisting of biotin, streptavidin and avidin, and a linker which connects the functional group to a backbone of the compound of Chemical Formula 1 therethrough;
m is an integer of 2˜10;
n is 0 or 1; and
R is selected from the group consisting of —H, —CH₃, —CH(CH₃)₂, —CH₂CH(CH₃)₂, —CHCH₃CH₂CH₃, —CH₂OH, —CHOHCH₃, —CH₂SH, —(CH₂)₂SCH₃, —CH₂COOH, —CH₂CONH₂, —(CH₂)₂COOH, —(CH₂)₂CONH₂, —(CH₂)₃CH₂NH₂, —(CH₂)₃NHCNHNH₂, 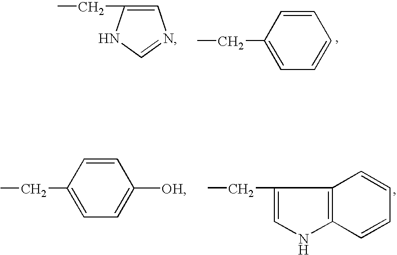
—CH₂CH₂CH₂— and —CH₂SSCH₂—.

2. The agent as set forth in claim 1, wherein the linker is selected from the group consisting of polyethylene glycols, DNA, C₁˜C₂₀ alkylene 1-ethyl-3-(3-dimethylaminopropyl)carbodiimide (EDC), carbonyldiimidazole (CDI), Sulfo-NHS (sulfosuccinimidyl), isocyanate derivatives, acylazide derivatives, N-hydroxysuccinimide (NHS), sulfonyl chloride derivatives, aldehyde derivatives and epoxy derivatives.

3. The agent as set forth in claim 1, wherein m is 2 and the amino acid moiety is selected from the group consisting of ALA-ALA, ARG-ARG, ARG-ASN, ASN-TYR, CYS-VAL, GLN-HIS, GLN-ILE, GLU-HIS, GLU-LYS, GLY-GLU, ILE-THR, LEU-ALA, LYS-LYS, MET-ALA, MET-APS, PHE-ASN, PRO-ALA, SER-ARG, SER-CYS, SER-LYS, SER-VAL, THR-GLN, THR-GLU, THR-LYS, TRP-ARG, TYR-ALA, VAL-ALA, VAL-HIS and GLU-HIS.

4. The agent as set forth in claim 1, binding to an H5 protein of avian influenza virus.

5. A compound for capturing or inhibiting avian influenza virus, represented by the following Chemical Formula 1: 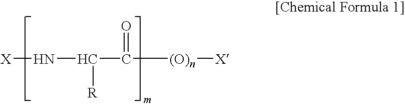
wherein
X and X′ are independently H; a functional group selected from the group consisting of biotin, streptavidin and avidin; or a functional moiety composed of a functional group selected from the group consisting of biotin, streptavidin and avidin, and a linker which connects the functional group to the backbone of the compound of Chemical Formula 1 therethrough, with a proviso that X and X′ are not H at the same time;
m is an integer of 2˜10;
n is 0 or 1; and
R is selected from the group consisting of —H, —CH₃, —CH(CH₃)₂, —CH₂CH(CH₃)₂, —CHCH₃CH₂CH₃, —CH₂OH, —CHOHCH₃, —CH₂SH, —(CH₂)₂SCH₃, —CH₂COOH, —CH₂CONH₂, —(CH₂)₂COOH, —(CH₂)₂CONH₂, —(CH₂)₃CH₂NH₂, —(CH₂)₃NHCNHNH₂, 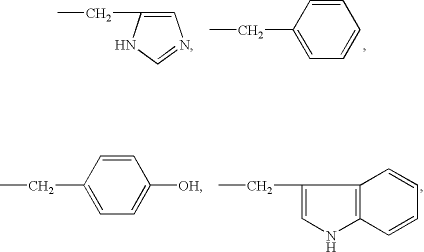
—CH₂CH₂CH₂— and —CH₂SSCH₂—.

6. The compound as set forth in claim 5, wherein the linker is selected from the group consisting of polyethylene glycols, DNA, C₁˜C₂₀ alkylene 1-ethyl-3-(3-dimethylaminopropyl)carbodiimide (EDC), carbonyldiimidazole (CDI), Sulfo-NHS (sulfosuccinimidyl), isocyanate derivatives, acylazide derivatives, N-hydroxysuccinimide (NHS), sulfonyl chloride derivatives, aldehyde derivatives, and epoxy derivatives.

7. A method for detecting avian influenza virus, comprising contacting a compound, represented by the following Chemical Formula 1, with a sample to be tested: 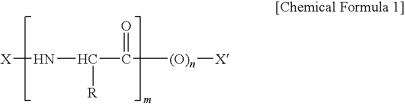
wherein
X and X′ are independently: H; a functional group selected from the group consisting of biotin, streptavidin and avidin; or a functional moiety composed of a functional group selected from the group consisting of biotin, streptavidin and avidin, and a linker which connects the functional group to a backbone of the compound of Chemical Formula 1 therethrough;
m is an integer of 2˜10;
n is 0 or 1; and
R is selected from the group consisting of —H, —CH₃, —CH(CH₃)₂, —CH₂CH(CH₃)₂, —CHCH₃CH₂CH₃, —CH₂OH, —CHOHCH₃, —CH₂SH, —(CH₂)₂SCH₃, —CH₂COOH, —CH₂CONH₂, —(CH₂)₂CONH₂, —(CH₂)₃CH₂NH₂, —(CH₂)₃NHCNHNH₂, 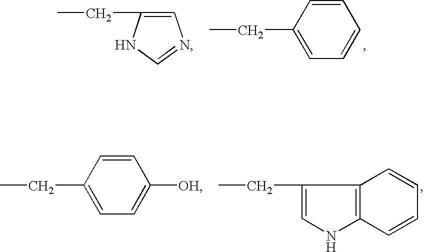
—CH₂CH₂CH₂— and —CH₂SSCH₂—.

8. The method as set forth in claim 7, wherein the contacting step comprises:
fixing either the compound of Chemical Formula 1 or the sample on a substrate; and
bringing the compound of Chemical Formula 1 into contact with the sample when it is fixed on the substrate and vice versa.

9. The method as set forth in claim 8, wherein the contacting step further comprises adding a label-conjugated secondary capturing material to the substrate when the sample is brought into contact with the compound of Chemical Formula 1 after the compound of Chemical Formula 1 is fixed on the substrate.

10. The method as set forth in claim 7, wherein the contacting step comprises:
fixing the compound of Chemical Formula 1 on nano- or microbeads; and
bringing the sample into contact with the fixed compound of Chemical Formula 1.

11. The method as set forth in claim 10, wherein the contacting step further comprises adding a label-conjugated secondary capturing material to the nano- or microbeads after the sample is brought into contact with the fixed compound of Chemical Formula 1.

12. The method as set forth in claim 10, wherein the nano- or microbeads are magnetic particles.

13. A biosensor for diagnosing avian influenza infection, comprising the agent of claim 1 as a detector or capturer of avian influenza virus.
